(19) United States
(12) Patent Application Publication
(10) Pub. No.: US 2024/0355444 A1
(43) Pub. Date: Oct. 24, 2024

(54) MODEL FOR DETERMINING IHC POSITIVITY

(71) Applicant: NEC Laboratories America, Inc., Princeton, NJ (US)

(72) Inventor: Eric Cosatto, Red Bank, NJ (US)

(21) Appl. No.: 18/623,576

(22) Filed: Apr. 1, 2024

Related U.S. Application Data
(60) Provisional application No. 63/460,717, filed on Apr. 20, 2023.
(60) Provisional application No. 63/460,706, filed on Apr. 20, 2023.

(51) Int. Cl. G16H 20/10; G06T 7/00; G06T 7/90; G16H 50/20

(52) U.S. Cl. CPC G16H 20/10; G06T 7/0012; G06T 7/90; G16H 50/20; G06T 2207/20081; G06T 2207/20084; G06T 2207/30024; G06T 2207/30096

(57) ABSTRACT

Methods and systems for diagnosing and treating cancer include performing color deconvolution on an input image, stained according to a second staining process, to generate channels that correspond to dyes used in a first staining process and dyes using in the second staining process. Channels that correlate with a channel used to train a machine learning model are combined to produce a single combined channel. The combined channel is processed using the machine learning model to identify tumor cells. A positivity index is determined based on an output of the machine learning model to aid in medical decision making. A patient's treatment is automatically adjusted based on an output of the machine learning model.

### RELATED APPLICATION INFORMATION

This application claims priority to U.S. Patent Application No. 63/460,706, filed on Apr. 20, 2023, and to U.S. Patent Application No. 63/460,717, filed on Apr. 20, 2023, incorporated herein by reference in their entirety.

### BACKGROUND

### Technical Field

The present invention relates to machine learning models and, more particularly, to models used for processing histological images.

### Description of the Related Art

Staining is a technique used in histopathology, where dyes are applied to a tissue to identify different structures. Images of tissue stained with such dyes can then be analyzed by a trained pathologist to identify potentially dangerous structures, such as cancerous tumors. Such diagnosis is the first step to providing life-saving treatments to patients.

### SUMMARY

A method for diagnosing and treating cancer includes performing color deconvolution on an input image of tissue, stained according to a second staining process, to generate image color channels that correspond to dyes used in a first staining process and dyes using in the second staining process. Channels that correlate with a channel used to train a machine learning model are combined to produce a single combined channel. The combined channel is processed using the machine learning model to identify tumor cells and non-tumor cells. A positivity index is determined based on an output of the machine learning model to aid in medical decision making. A patient's treatment is automatically adjusted based on an output of the machine learning model.

A system for diagnosing and treating cancer includes a hardware processor and a memory that stores a computer program. When executed by the hardware processor, the computer program causes the hardware processor to perform color deconvolution on an input image of tissue, stained according to a second staining process, to generate image color channels that correspond to dyes used in a first staining process and dyes using in the second staining process, to combine channels that correlate with a channel used to train a machine learning model to produce a single combined channel, to process the combined channel using the machine learning model to identify tumor cells and non-tumor cells, to determine a positivity index based on an output of the machine learning model to aid in medical decision making, and to automatically adjust a patient's treatment based on an output of the machine learning model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is an image of an exemplary tissue stain, in accordance with an embodiment of the present invention;

FIG. 2 is a block/flow diagram of a method of training and using an image processing model that is trained on images stained with a first technique, and that is capable of processing images stained with a second technique, in accordance with an embodiment of the present invention;

FIG. 3 is a block/flow diagram of a method of processing an image with a machine learning model to determine a positivity index, in accordance with an embodiment of the present invention;

FIG. 4 is a block diagram of an exemplary healthcare facility where tissue staining and analysis is performed, in accordance with an embodiment of the present invention;

FIG. 5 is a block diagram of a computing device that can perform model training and image processing, in accordance with an embodiment of the present invention;

FIG. 6 is a diagram of an exemplary neural network architecture that can be used for image processing, in accordance with an embodiment of the present invention; and

FIG. 7 a diagram of an exemplary deep neural network architecture that can be used for image processing, in accordance with an embodiment of the present invention.

### DETAILED DESCRIPTION OF PREFERRED EMBODIMENTS

Histological staining can be used to diagnose a variety of health conditions, by making it possible to analyze the cellular structure of different kinds of tissue. While histological images are often analyzed by an expert, they can also be used as the input to a machine learning model. Such a model can be trained on a set of previously analyzed and labeled histological images to automatically identify health conditions indicated by the images.

There are various types of staining that are used in histopathology to visualize and differentiate tissues and cells under a microscope. One such type is hematoxylin and eosin (H&E) staining, where hematoxylin stains the nuclei of cells a blue-purple color, while eosin stains the cytoplasm and extracellular matrix of the tissue pink. H&E staining may be used to visualize the general tissue architecture, cell morphology, and abnormalities such as inflammation, necrosis, and cancer. H&E images thus include a mix of two colors: blue-purple and pink.

In clinical practice, a pathologist may use H&E slides identify tumor regions and may order further stains to assess the aggressiveness of the tumor or to discover whether it includes particular mutations.

Another type of staining is immunohistochemistry (IHC) staining, which involves the use of antibodies that are labeled with a detectable marker, such as a fluorescent dye or an enzyme. When the antibody binds to its target antigen in the tissue section, the labeled marker produces a visible signal that can be visualized under a microscope. IHC staining is used to identify the presence of specific proteins in tissues, such as tumor markers or markers of inflammation. An example of such a marker is DAB (3,3′-diaminobenzidine), which exhibits a brown color. In addition, IHC samples may be counter-stained with hematoxylin. Thus, IHC may include two colors, such as blue-purple and brown.

For many IHC markers, a positivity index is calculated to quantitatively assess the level of expression in a tissue sample. The positivity index may be expressed as the percentage of positively staining cells relative to the total number of cells within the sample. However, the index may be calculated on only the tumor cells. This is because some tissues may have many normal cells, which would affect the accuracy of the index.

There are many immunohistochemical (IHC) markers that are used in clinical and research settings to diagnose and characterize different types of cancers and other diseases. An example of an IHC marker is the Ki-67 protein, which is a marker of cellular proliferation. The Ki-67 positivity index is a measure of the proportion of actively dividing cells in a given tissue sample. Ki-67 may be used as a prognostic marker in cancer diagnosis and treatment. A further example of an IHC marker is ER/PR (estrogen receptor/progesterone receptor). These markers are used to determine the hormonal status of breast cancer and guide treatment decisions. A further example of an IHC marker is HER2 (human epidermal growth factor receptor 2). This marker is used to identify breast cancer patients who may benefit from targeted HER2 therapy.

Stained histological images may be used as part of a training dataset for a machine learning model. For example, such a model may have the function of classifying an input image to indicate whether the image shows cancerous tissue. In some cases, a neural network model can calculate the IHC positivity index of tumor cells directly from an IHC-stained image. Such a neural network model can be trained using supervision from labeled H&E-stained images and can be applied directly to IHC images.

Once a given tissue sample is classified as being normal or tumorous, tumor cells can be classified as IHC-positive or IHC-negative directly by sampling a channel of an IHC color deconvolution at the location of detected tumor cells.

A model that was trained using H&E-stained data and labels may be re-trained for use with IHC-stained images. Thus, a model which was originally trained to detect cell nuclei on H&E-stained images may be retrained so that it can detect cell nuclei on IHC-stained images. The training reuses the image and labels of the original model, thereby avoiding the cost of manual relabeling.

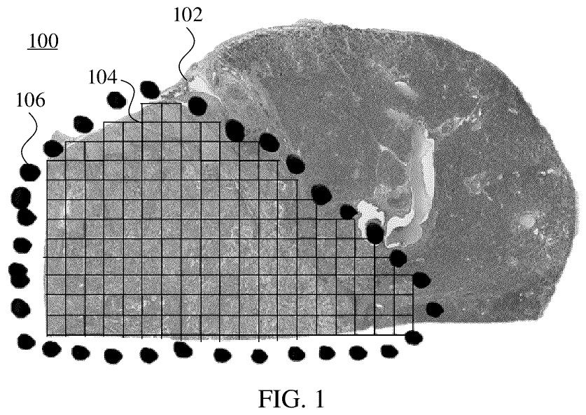

Referring now to FIG. 1, an exemplary stained slide image 100 is shown. The slide image 100 includes the scanned tissue image 102. A number of tiles 104 have been identified within the image, for example dividing the image 100 into regions that can be processed separately, for example by applying a grid over the scanned tissue image 102, with pixels falling within each respective grid making up a respective tile. A set of markers 106 may also be used, having been provided by a human operator, to limit the extent of the tiles to a region of interest, such that tiles 104 are only determined within a boundary that is established by the markers 106.

Each tile 104 may be separately processed, for example using parallel processing across multiple processors or processor cores. The total number of tiles may be divided by the number of available processing threads to form sets of tiles. Each set may be processed in serial by a respective thread.

The image 100 will show different features depending on the type of staining that is used. As noted above, H&E staining will produce an image of blue-purple and pink structures, while IHC staining may produce blue-purple and brown (or some other appropriate marker color). An H&E image may include three channels of information, for example using a color camera to generate red, green, and blue channels. As will be described in greater detail below, color deconvolution can be used to convert this three-channel information to a single channel representing the hematoxylin stain alone. Similarly, a three-channel IHC image may be deconvoluted to produce two channels that represent the hematoxylin stain and the DAB stain. Since both hematoxylin and DAB may stain the nuclei of cells, those channels can be combined into one channel that corresponds semantically to the hematoxylin channel in the H&E image. A model trained on H&E-stained images may therefore function correctly when using IHC-stained images in this manner, without retraining the model for IHC-stained images.

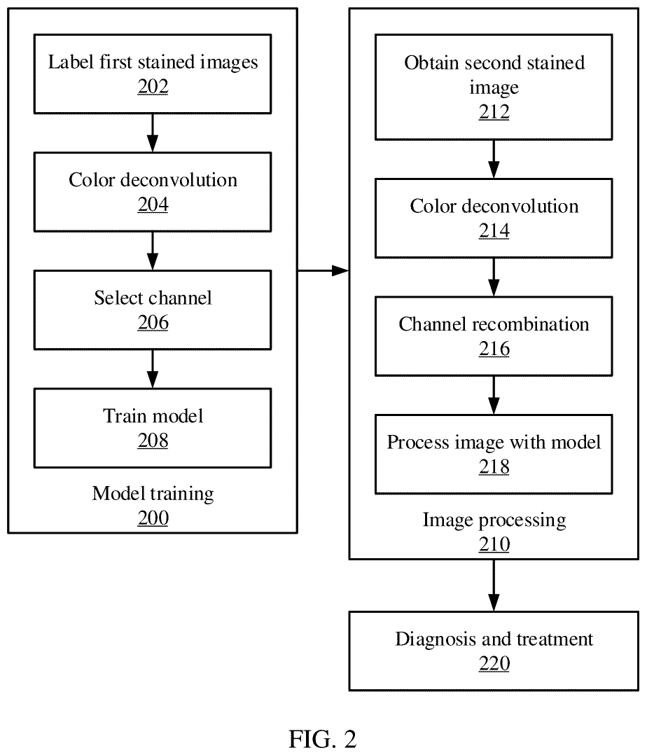

Referring now to FIG. 2, a method for training and using a machine learning model is shown. Three stages are shown, including model training 200, image processing 210, and diagnosis and treatment 220. Each of these stages may be performed in the same location/system, or they may be performed separately at different locations and by different entities.

Training the model is performed based on training data with a first type of staining (e.g., H&E staining). Block 202 labels the first stained images in accordance with a task. The task may include, for example, tumor cell counting, tissue classification, or any other appropriate task that may be performed using histological images. As noted above, the image may be broken up into tiles, which may be processed independently from one another, or they may be whole images.

Block 204 then performs color deconvolution on the first stained images. Color deconvolution converts red-green-blue (RGB) image information from the first stained images into a set of three channels, with each channel representing hematoxylin (H), eosin (E), and DAB (D) respectively. Any appropriate color deconvolution technique may be used by block 204.

Thus, the three-channel RGB first stained images are converted into three-channel HED images. In an HED image, each pixel of the H channel indicates the amount of hematoxylin dye at that pixel, each pixel of the E channel represents the amount of eosin dye at that pixel, and each pixel of the D channel represents an mount of the DAB marker at that pixel.

Since the H&E-stained images do not include the DAB staining, the D channel is empty in the output of this deconvolution. Further, since IHC staining does not use eosin, the E channel of the deconvolution may be ignored. Thus block 206 selects the H channel for use in training, producing respective first single-channel images that correspond to the first stained images. Block 208 trains a machine learning model to perform the task based on the first single-channel images.

The machine learning model may be any appropriate model, such as a neural network model that makes use of convolutional neural networks. The model may accept an image as input and produce an output. Training may be performed in a supervised fashion, using the labels of the first stained images corresponding to respective first single-channel images.

Once the model is trained, and has been deployed, it may be used to perform inferences during image processing 210. Block 212 obtains a second image that is stained with a different staining technique, for example using IHC staining. Block 214 performs color deconvolution on the three-channel IHC-stained image to produce a three-channel HED image.

In this case, the IHC image includes the H channel and the D channel, but the E channel is empty because IHC staining does not use eosin. Because the model is trained with only one channel, the H and D channels are merged into a single channel in channel recombination 216. Any appropriate technique for recombination can be used. For example, P=max (H, D) can be used where a recombined pixel's value is taken as the maximum value between the H and D channels for the pixel. Another approach is to use a linear combination P=kH+ (1−k) D to mix the values of the H and D channels, with k being a weighting parameter between zero and one.

Block 218 processes the image using the trained neural network, for example using the recombined channel. For example, the model may output two sets of cells, normal cells and tumor cells. For each identified cell, the model may output a location of the cell within the image. One exemplary architecture for the model is a fully convolutional U-net model, which detects and classifies the cells as described in greater detail below.

The recombined image may then be used as a single-channel input to the trained model, which performs the designated task and generates a corresponding output. For example, in a tumor cell classification task, the model may output a list of tumor and non-tumor cells present in the second stained image. In a cancer detection task, the model may output a determination of whether the tissue shown in the second stained image is cancerous.

Based on the output of the model, diagnosis and treatment 220 are performed. Diagnosis may include post-processing of the raw output of the model, for example using peak detection to identify and classify objects such as cell nuclei within the model's output maps. Diagnosis may include identifying a type of cancer or other illness, while treatment may include the administration of one or more drugs, surgeries, or therapies to treat the illness. The diagnosis may be performed automatically, for example in the case of a cancer detection task that indicates that a patient has cancer. The treatment may also be performed automatically, for example in the administration of an appropriate dosage of an anti-cancer medication.

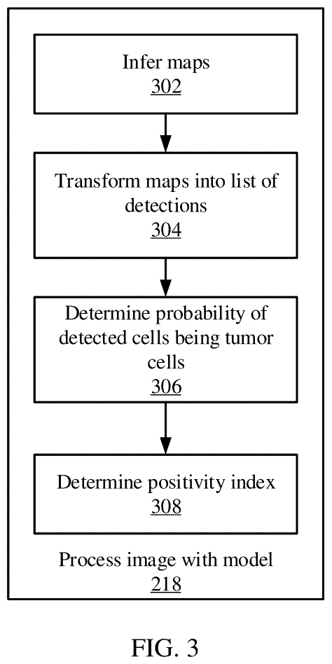

Referring now to FIG. 3, additional detail on processing the image with a pre-trained model in block 218 is shown. Block 302 uses the model to infer two maps from the input map, for example using the combined HD channels. A first map represents the probability that each pixel represents the center of a tumor cell nucleus, while a second map represents the probability that each pixel represents the center of a non-tumor cell nucleus. Block 304 transforms the maps into a list of detections using a peak detector, marking the peaks on the maps. Block 306 determines a score representing a probability of each detection being a tumor cell, for example using a softmax function that is applied to the two map values at each peak.

Using only tumor cells, the D channel can be sampled at each cell location to provide an IHC score based on the intensity of the D channel at the sampling location. A threshold may be obtained on a validation set. Tumor cells for which the IHC score is above the threshold may be identified as tumor-positive cells in diagnosis 220, while tumor cells for which the IHC score is below the threshold may be identified as tumor_negative. Non-tumor cells for which the IHC score is above the threshold may be identified as non-tumor_positive and non-tumor cells for which the IHC score is below the threshold may be identified as non-tumor_negative. The IHC positivity index is obtained from tumor cells only and can therefore be computed in block 308 as:

tumor_positive/(tumor_positive+tumor_negative)

Any appropriate process may be used to determine the threshold value. One exemplary approach is to obtain a validation set of IHC images where all nuclei have been labeled as either non-tumor, tumor-negative, or tumor-positive. Then a threshold value may be determined that minimizes the error between the labels and the model-classified cells.

Only the D channel of the IHC image carries information about the relevant immune marker (e.g., Ki-67), while the H channel carries information about nuclei morphology. As a result, classification to distinguish between tumor cells and non-tumor cells may be performed on the H channel alone, while the D channel may be sampled to calculate the IHC score.

The IHC positivity index may be used to help healthcare personnel in diagnosing specific aspects of a tumor. For example, a Ki-67 positivity index indicates the rate at which tumor cells are growing. In such embodiments the dye binds to the Ki-67 protein, which is involved in cell proliferation. This can help to identify fast-growing tumors that need to be treated aggressively, for example with early surgical and chemotherapeutic treatments being applied.

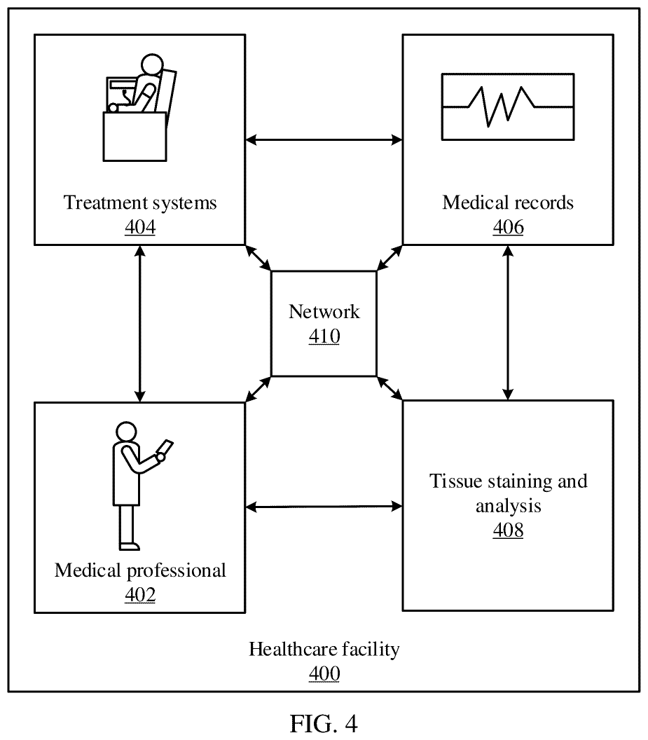

Referring now to FIG. 4, a diagram of tissue staining and analysis is shown in the context of a healthcare facility 400. Tissue staining and analysis 408 may be used to analyze tissue samples that have been stained according to different staining processes. For example, an analysis model may be trained on H&E training data, and so may accept H&E-stained tissue sample images, but may also accept IHC-stained tissue sample images after appropriate processing.

The healthcare facility may include one or more medical professionals 402 who review information from a patient's medical records 406 to determine their healthcare and treatment needs. These medical records 406 may include tissue samples collected from the patient and stained according to a staining process that the model can accept. Treatment systems 404 may furthermore monitor patient status to generate medical records 406 and may be designed to automatically administer and adjust treatments as needed.

Based on information drawn from the tissue staining and analysis 408, the medical professionals 402 may then make medical decisions about patient healthcare suited to the patient's needs. For example, the medical professionals 402 may make a diagnosis of the patient's health condition and may prescribe particular medications, surgeries, and/or therapies.

The different elements of the healthcare facility 400 may communicate with one another via a network 410, for example using any appropriate wired or wireless communications protocol and medium. Thus tissue staining and analysis 408 receives tissue samples from the medical records 406, updates the medical records 406 with the output of the trained model, and may coordinate with treatment systems 404 in some cases to automatically administer a treatment.

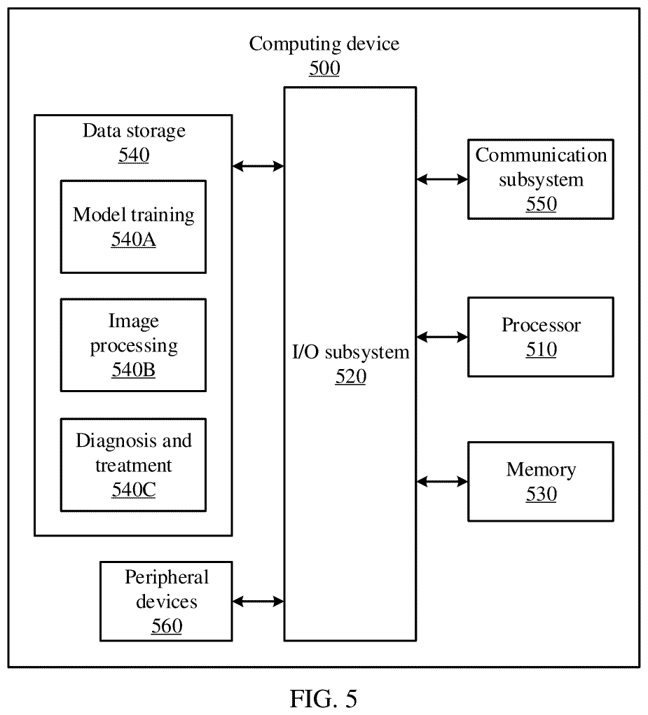

Referring now to FIG. 5, an exemplary computing device 500 is shown, in accordance with an embodiment of the present invention. The computing device 500 is configured to perform tissue analysis.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for training a model, 540B for performing image processing, and/or 540C for performing diagnosis and treatment. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

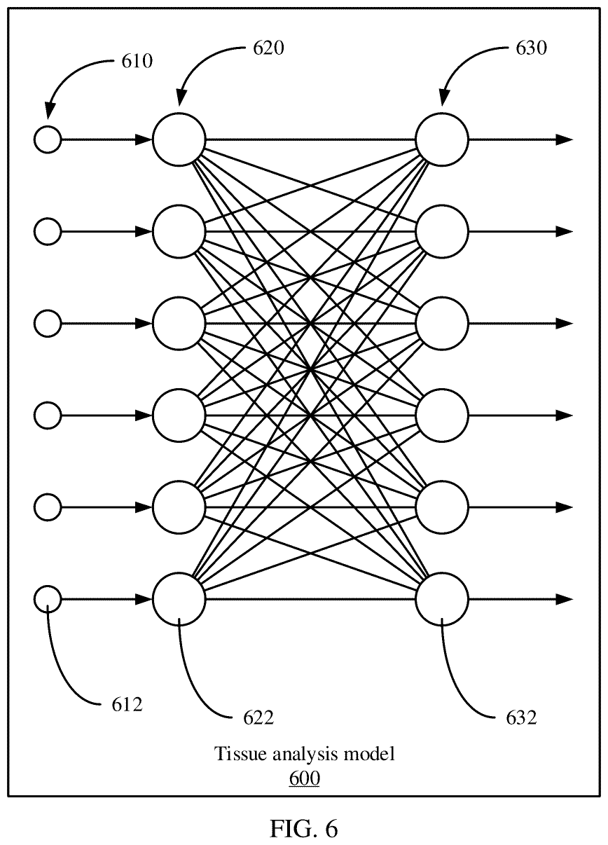

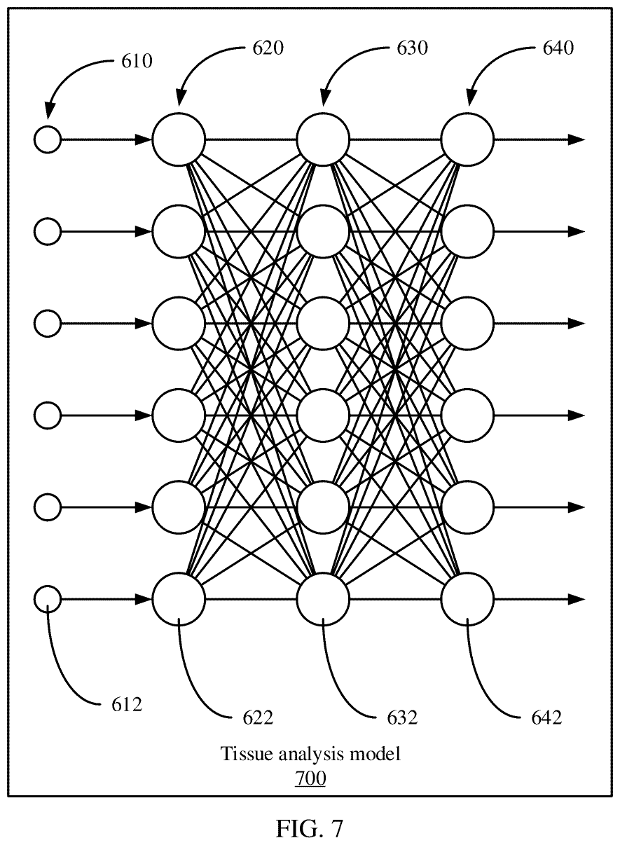

Referring now to FIGS. 6 and 7, exemplary neural network architectures are shown, which may be used to implement parts of the present models, such as the tissue analysis model 600/700. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for diagnosing and treating cancer, comprising:
performing color deconvolution on an input image of tissue, stained according to a second staining process, to generate a plurality of image color channels that correspond to dyes used in a first staining process and dyes using in the second staining process;
combining channels of the plurality of channels that correlate with a channel used to train a machine learning model to produce a single combined channel;
processing the combined channel using the machine learning model to identify tumor cells and non-tumor cells;
determining a positivity index based on an output of the machine learning model to aid in medical decision making; and
automatically adjusting a patient's treatment based on an output of the machine learning model.

2. The method of claim 1, wherein the first staining process is hematoxylin and eosin (H&E) staining and the second staining process is immunohistochemistry (IHC) staining.

3. The method of claim 2, wherein the plurality of channels include a hematoxylin (H) channel, an eosin (E) channel, and a 3,3′-diaminobenzidine (D) channel.

4. The method of claim 3, wherein combining channels of the plurality of channels includes combining the H and D channels.

5. The method of claim 4, wherein combining channels includes setting pixel values of the combined channel according to maximum values of corresponding pixels in the H and D channels.

6. The method of claim 4, wherein combining channels includes setting pixel values of the combined channel according to a linear combination of corresponding pixels in the H and D channels.

7. The method of claim 4, wherein each identified cell is assigned an IHC score by sampling a corresponding location of the D channel.

8. The method of claim 7, wherein each identified cell is determined to be IHC positive if its IHC score is above a threshold value and the IHC is negative otherwise.

9. The method of claim 8, wherein the positivity index is determined as a ratio between a number of tumor-positive cells to a total number of tumor cells.

10. The method of claim 1, wherein automatically adjusting the patient's treatment includes automatically administering an anti-cancer medication responsive to a determination that the input image indicates a tumor.

11. A system for diagnosing and treating cancer, comprising:
a hardware processor; and
a memory that stores a computer program which, when executed by the hardware processor causes the hardware processor to:
perform color deconvolution on an input image of tissue, stained according to a second staining process, to generate a plurality of image color channels that correspond to dyes used in a first staining process and dyes using in the second staining process;
combine channels of the plurality of channels that correlate with a channel used to train a machine learning model to produce a single combined channel;
process the combined channel using the machine learning model to identify tumor cells and non-tumor cells;
determine a positivity index based on an output of the machine learning model to aid in medical decision making; and
automatically adjust a patient's treatment based on an output of the machine learning model.

12. The system of claim 11, wherein the first staining process is hematoxylin and eosin (H&E) staining and the second staining process is immunohistochemistry (IHC) staining.

13. The system of claim 12, wherein the plurality of channels include a hematoxylin (H) channel, an eosin (E) channel, and a 3,3′-diaminobenzidine (D) channel.

14. The system of claim 13, wherein the computer program further causes the hardware processor to combine the H and D channels.

15. The system of claim 14, wherein the computer program further causes the hardware processor to set pixel values of the combined channel according to maximum values of corresponding pixels in the H and D channels.

16. The system of claim 14, wherein the computer program further causes the hardware processor to set pixel values of the combined channel according to a linear combination of corresponding pixels in the H and D channels.

17. The system of claim 14, wherein the computer program further causes the hardware processor to set an IHC score for each identified cell by sampling the corresponding location of the D channel.

18. The system of claim 17, wherein the computer program further causes the hardware processor to determine each identified cell to be IHC positive if its IHC score is above a threshold value and IHC negative otherwise.

19. The system of claim 18, wherein the positivity index is determined as a ratio between a number of tumor-positive cells to a total number of tumor cells.

20. The system of claim 11, wherein the computer program further causes the hardware processor to automatically administer an anti-cancer medication responsive to a determination that the input image indicates a tumor.
